(19) United States
(12) Patent Application Publication
(10) Pub. No.: US 2004/0014129 A1
(43) Pub. Date: Jan. 22, 2004

(54) Amplification primer pairs and use thereof

(76) Inventor: Bob D. Brown, Encinitas, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/621,009

(22) Filed: Jul. 15, 2003

Related U.S. Application Data
(63) Continuation of application No. 09/932,129, filed on Aug. 16, 2001, now abandoned.
Application No. 09/932,129 is a continuation of application No. PCT/US00/09230, filed on Apr. 7, 2000.
(60) Provisional application No. 60/128,378, filed on Apr. 8, 1999.

(51) Int. Cl. C12Q 1/68; C07H 21/04; C12P 19/34

(52) U.S. Cl. 435/6; 435/91.2; 536/24.3

(57) ABSTRACT

Amplification primer pairs comprising an oligonucleotide anchor and primer. The anchor has a nucleic acid chemistry which is not a substrate for reverse transcriptases and DNA polymerases, and a 3′-end which is not capable of priming DNA synthesis. The primer has a nucleic acid chemistry that is a substrate for reverse transcriptases and DNA polymerases. The anchor and the primer each include regions of nucleotide complementarity which are capable of associating with each other to form a stem structure. The stem structure includes a nucleotide sequence which is complementary to a universal primer. One primer pair (reverse pair) is used for first strand synthesis, while the second primer pair (forward pair) is used for second strand synthesis. The universal primers sustain the amplification reaction and produce the majority of the final product.

### CROSS-REFERENCE TO RELATED APPLICATIONS

This application is a continuation of and claims priority to U.S. patent application Ser. No. 09/932,129, filed Aug. 16, 2001, which is a continuation of and claims priority to International Application No. PCT/US00/09230, filed Apr. 7, 2000, designating the United States of America and published in English, which claims the benefit of priority under 35 U.S.C. § 119(e) to U.S. Provisional Application No. 60/128,378, filed Apr. 8, 1999; all of which are hereby expressly incorporated by reference in their entireties.

### BACKGROUND OF THE INVENTION

1. Field of the Invention

The present invention relates to two-component combinatorial PCR primer pairs and their use in amplifying nucleic acid sequences.

2. Description of the Related Art

The polymerase chain reaction (PCR) is a DNA amplification method in which oligonucleotide primers complementary to opposite ends of a gene sequence are used to amplify the sequence by repeated cycles of denaturation, annealing and extension in the presence of a DNA polymerase having activity at elevated temperatures. However, this method relies on knowing the sequences at the opposite ends of the gene in order to design primers which can hybridize to these regions. Thus, each time a gene is to be amplified, different oligonucleotide primers must be synthesized. Alternatively, if one wishes to have primers on hand, a standing library of millions or billions of conventional oligonucleotides would be needed to successfully target and amplify each of the approximately 100,000 human genes. An ordered library of millions of PCR primers is beyond the chemical, physical and organizational tools currently available.

There is a need for a pre-made PCR primer library containing reasonable numbers of primers capable of binding to every target sequence present in the genome. The present invention addresses this need.

### SUMMARY OF THE INVENTION

One embodiment of the present invention is an amplification primer pair comprising an oligonucleotide anchor and primer, the anchor having a nucleic acid chemistry which is not a substrate for reverse transcriptases and DNA polymerases, and/or having a 3′-end which is not capable of priming DNA synthesis; the primer having a nucleic acid chemistry that is a substrate for reverse transcriptases and DNA polymerases; the anchor and the primer each including a region of complementary nucleotides which are capable of associating with each other to form a stem structure which includes a region which is complementary to a universal primer. Preferably, the anchor sequence and the stem regions are connected by a flexible linker. In one aspect of this preferred embodiment, the flexible linker is selected from the group consisting of polyethylene glycol, polypropylene glycol, polyethylene, polypropylene, polyamides and polyesters. Preferably, the primer comprises a tail region which extends beyond the length of the stem region of the anchor. In one aspect of this preferred embodiment, the primer comprises one or more modified bases. In another aspect of this preferred embodiment, the anchor comprises one or more modified backbone linkages. Advantageously, the anchor and said primer are each between 6 and 24 bases in length. The present invention also provides a method for amplifying a nucleic acid sequence, comprising the steps of: combining a nucleic acid moleulce with a forward anchor (FA), forward primer (FP), reverse anchor (RA), reverse primer (RP), forward universal primer (FUP) and reverse universal primer (RUP), wherein the FA/FP form a first primer pair and the RA/RP form a second primer pair via association of their complementary stem regions, wherein the FUP is complementary to the FA/FP stem region, and wherein the RUP is complementary to the RA/RP stem region wherein the primer pairs are selected to flank the RNA sequence; and amplifying said nucleic acid sequence via enzyme-mediated amplification. Advantageously, the nucleic acid sequence encodes a therapeutic gene product. Preferably, the nucleic acid sequence is DNA or RNA. In one aspect of this preferred embodiment, the enzyme-mediated amplification is PCR amplification.

### BRIEF DESCRIPTION OF THE DRAWINGS

FIGS. 1A and 1B are schematic diagrams of forward and reverse amplification primer pairs, respectively. Each primer pair is generated from a library of individual oligonucleotides which are combined to form the primer pairs shown in the figure. One component of the primer pair is called the anchor (forward anchor=FA; reverse anchor=RA), and the other component is called the primer (forward primer=FP; reverse primer=RP). The two components are non-covalently bonded by a region of complementary oligonucleotides called the stem. The anchors may be linked to the stems by a linker. The forward universal primer (FUP) and reverse universal primer (RUP) are complementary to the forward and reverse stems, respectively. The primer pairs are tailored to amplify a specific gene sequence by choosing the appropriate individual oligonucleotide sequences. The reverse primer primes first strand cDNA synthesis, and the forward primer primes second strand cDNA synthesis.

FIG. 2 is a schematic diagram of an amplification primer pair showing the optional linker, stem, optional tail and universal primer.

FIG. 3 is a schematic diagram of the method of the invention using amplification primer pairs generated from a library of individual oligonucleotides. RA and FA are oligonucleotides comprising a plurality of modified bases and linkages for maximal binding affinity and are not capable of priming the nucleic acid amplification reaction (i.e. do not contain a 3′-OH group). These components may include a flexible linker. RP and FP serve as the amplification primers and are incorporated into the resulting DNA molecule. First strand cDNA synthesis is primed by RP, and second strand cDNA synthesis is primed by FP. FA and RA are displaced during the amplification reaction. RUP and FUP are universal primers complementary to a portion of RP and FP, respectively, which are incorporated into the amplified cDNA. RUP and FUP primer reverse and forward product strand synthesis, respectively. The final product is a double stranded amplicon containing RUP and FUP.

### DETAILED DESCRIPTION OF THE PREFERRED EMBODIMENTS

The present invention provides amplification primer pairs for use in amplifying any DNA sequence of interest. These primer pairs can be prepared quickly, using a feasible number of pre-synthesized components present in a primer library, and are used for nucleic acid synthesis and detection. The library of components is suitable for forming any desired amplification primer pair on demand. To amplify any desired nucleic acid sequence, two amplification primer pairs are used, each being generated from individual oligonucleotide components. All possible 6-mer, 8-mer, 12-mer or other length oligonucleotides are synthesized by conventional automated DNA sequencing and include a region which is complementary to a second set of 6-mers, 8-mers, 12-mers (or other length oligomers) so that when two such primers are combined in vitro, they self-assemble via hydrogen bonding of their complementary regions (Watson-Crick base pairing). In one embodiment of the invention, the anchor and primer oligonucleotides are each between 6 and about 24 bases in length.

All possible 8-mers, for example, can be represented in a modestly-sized library of 4⁸ (65,536) oligonucleotides. All possible 16-mers, on the other hand, would require an enormous library of 4¹⁶ (4×10⁹) oligonucleotides.

The term “oligonucleotide” refers to a molecule consisting of DNA, RNA or DNA/RNA hybrids.

The term “oligonucleotide analog” refers to a molecule comprising an oligonucleotide-like structure, for example having a backbone and a series of bases, wherein the backbone and/or one or more of the bases can be other than the structures found in naturally-occurring DNA and RNA. “Non-natural” oligonucleotide analogs include at least one base or backbone structure that is not found in natural DNA or RNA. Exemplary oligonucleotide analogs include, but are not limited to, DNA, RNA, phosphorothioate oligonucleotides, peptide nucleic acids (PNA), methoxyethyl phosphorothioates, oligonucleotide containing deoxyinosine or deoxy 5-nitroindole, and the like.

The term “backbone” as used herein refers to a generally linear molecule capable of supporting a plurality of bases attached at defined intervals. Preferably, the backbone will support the bases in a geometry conducive to hybridization between the supported bases of a target polynucleotide.

The term “non-naturally occurring base” refers to a base other than A, C, G, T and U, and includes degenerate and universal bases as well as moieties capable of binding specifically to a natural base or to a non-naturally occurring base. Non-naturally occurring bases include, but are not limited to, propynylcytosine, propynyluridine, diaminopurine, 5-methylcytosine, 7-deazaadenosine and 7-deazaguanine.

The term “universal base” refers to a moiety that may be substituted for any base. The universal base need not contribute to hybridization, but should not significantly detract from hybridization. Exemplary universal bases include, but are not limited to, inosine, 5-nitroindole and 4-nitrobenzimidazole.

The term “degenerate base” refers to a moiety that is capable of base-pairing with either any purine, or any pyrimidine, but not both purines and pyrimidines. Exemplary degenerate bases include, but are not limited to, 6H, 8H-3,4-dihydropyrimido[4,5-c][1,2]oxazin-7-one (“P”, a pyrimidine mimic) and 2-amino-6-methoxyaminopurine (“K”, a purine mimic).

The term “target polynucleotide” refers to DNA or RNA, for example as found in a living cell, with which a primer pair is intended to bind or react.

The term “library” refers to a collection of components that can be joined to form a variety of different molecules. In the practice of the invention, a library comprises at least two sets of oligonucleotides, designed such that oligomers of the first set can couple to oligomers of the second set. This coupling preferably occurs spontaneously on addition of the two oligomers.

The term “flexible linker” as used herein refers to a reactive chemical group that is capable of covalently attaching a binding domain to a coupling moiety. These linkers relieve stress that might otherwise result from interposing the coupling moieties between two binding domains that bind to adjacent regions of target nucleic acid. The flexible linker is preferably selected to be flexible, hydrophilic, and of sufficient length that the bulk of the coupling moieties does not interfere with hybridization., RNase recognition, and/or RNase activity on the complex. The linker may be connected to the terminal base of the binding domain, or can be connected one or more bases from the end. Suitable flexible linkers are typically linear molecules in a chain of at least one or two atoms, more typically an organic polymer chain of 1 to 12 carbon atoms (and/or other backbone atoms) in length. Exemplary flexible linkers include polyethylene glycol, polypropylene glycol, polyethylene, polypropylene, polyamides, polyesters and the like.

The term “modified backbone linkage” refers to internucleoside linkages other than phosphodiester linkages. Examples of these linkages include phosphorothioates, phosphorodithioates, methylphosphonates, morpholinos, MMI, peptide nucleic acids (PNA), 3′-amidates and the like.

The term “stem” as used herein refers to the structure formed by coupling two oligonucleotide or oligonucleotide analog coupling moieties.

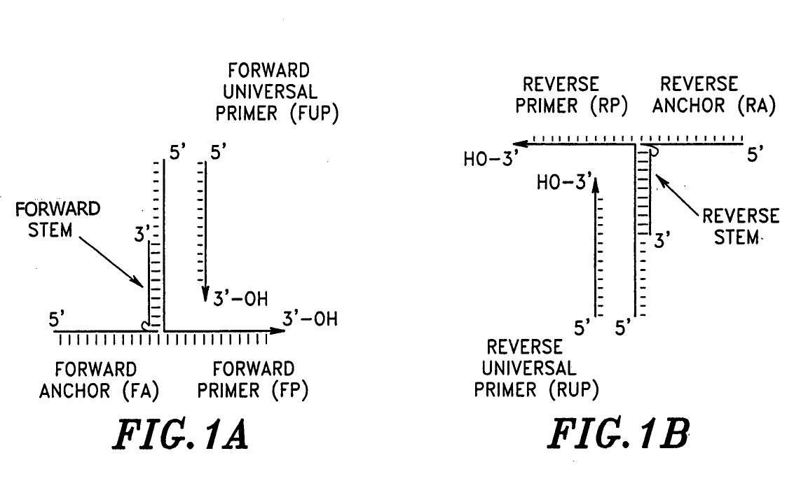

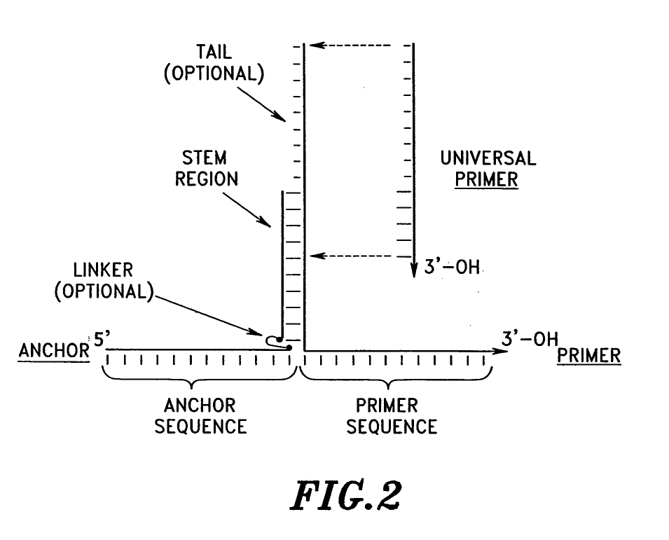

Referring to FIGS. 1A-1B, two primer pairs are used: a forward pair and a reverse pair. Each pair comprises an anchor and a primer which are bonded via complementary base pairing in the stem region. The sequences of FA, FP, RA and RP are chosen from a primer library on the basis of the exact nucleic acid sequence or gene sequence to be amplified. The FUP and RUP primer sequences are not gene-specific. These sequences of these primers are based on the common forward stem and reverse stem sequences, respectively. FUP hybridize to all or part of the FP stem region, and RUP hybridizes to all or part of the RP stem region. The “tail” region shown in FIG. 2 is optional. The FA and FP associate via base hybridization of the forward stem sequence, and RA and RP associate in the same manner. The stem sequences are too long for the small, overlapping complimentary regions in the FUP and RUP to interfere. Any FA can form a stem (hybridize to form a short duplex region) with any FP due to complementary stem sequences. Stem formation is not possible between any forward and reverse library members. Thus, FA will only pair with FP, and RA will only pair with RP.

The anchor components of the primer pairs preferably contain one or more modified bases and/or degenerate bases and/or universal bases, and may also contain one or more modified backbone linkages, to result in maximum affinity binding to a nucleotide sequence of interest. The anchors can have any nucleic acid chemistry (modified bases and/or linkages) which are not substrates for reverse transcriptases or DNA polymerases, and/or do not contain a chemical group such as a 3′-OH group, which is capable of priming DNA synthesis. In contrast, the primer components are much more “DNA-like” and contain substantially naturally-occurring oligonucleotides (A, G, T, C, U) and phosphodiester linkages because they must serve as a template for reverse transcriptase and DNA polymerase. The primer components may comprise any nucleic acid chemistry (combination of natural bases, unnatural bases, phosphodiester linkages, modified backbone linkages) which acts as a substrate for reverse transcriptase and DNA polymerase. The ability of any desired oligonucleotide or oligonucleotide analog to act as a substrate for reverse transcriptase and DNA polymerase can be readily determined by a person of ordinary skill in the art.

The anchor sequence and primer sequence may be joined to the stem region by an optional flexible linker (FIG. 2). A linker is likely to be more appropriate for anchors to relieve strain, increase binding affinity and improve the priming/polymerase substrate activity of the assembled anchor/primer complexes. Primers can also comprise linkers, although one should be used that can maintain polymerase activity.

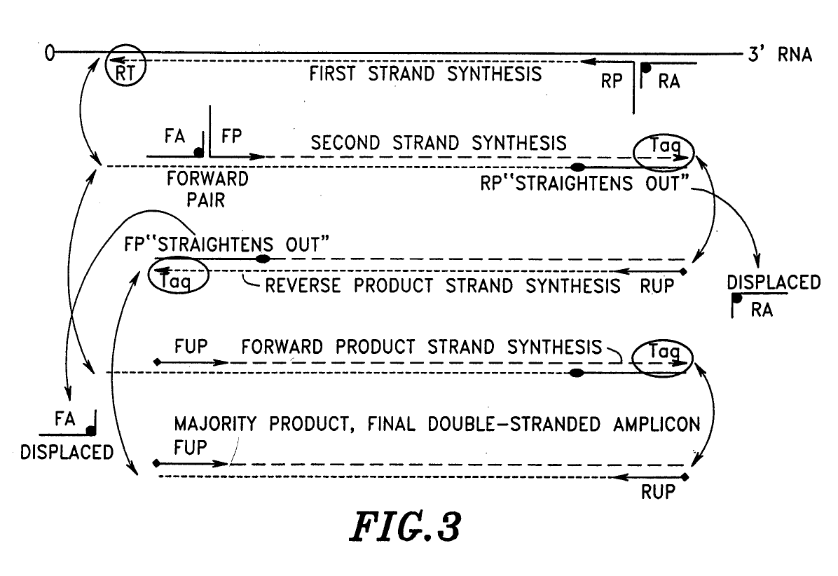

With knowledge of the sequence to be amplified, the appropriate complementary FA, FP, RA, and RP oligonucleotides are selected from the library and combined to form primers. The gene sequence-specific amplification primer pairs are mixed in complete amplification (e.g. PCR) reaction buffer, including the universal primers. The ratio of FA/FP and RA/RP is typically about 1 to 10 or less. Thus, FA/FP and RA/RP typically only catalyze first and second strand cDNA synthesis. RA/RP is used for first strand synthesis, while FA/FP is used for second strand synthesis (FIG. 3). In contrast, FUP and RUP sustain the amplification reaction and produce the majority of the final amplicon product. The FA and FP immediately combine only with each other via base hybridization of the forward stem sequence, and the RA and RP combine only with each other in the same manner.

The template RNA mixture to be amplified, typically total cellular RNA or poly(A)+ RNA, is added to the reaction and the target RNA is bound by the RA/RP complex based on the hybridization of the gene sequence-specific sequence formed by the RA/RP complex assembly. Typically, a combined reverse transcriptase/thermostable DNA polymerase enzyme mix (e.g. Taq polymerase) is added to the reaction which is incubated at a temperature suitable for reverse transcription of the RNA (generally 37-55° C.) to produce the first cDNA strand. First-strand cDNA synthesis is primed by the RA/RP structure, and the 3′-OH group of RP is extended by reverse transcriptase. RP is consumed by the reaction and becomes the 5′-end of the first strand cDNA product (FIG. 3). RA is displaced by second-strand cDNA synthesis and does not participate in subsequence rounds of amplification.

Second strand cDNA synthesis is primed by FA/FP, and the 3′-OH of FP is extended by DNA polymerase (thermostabile or otherwise). FP is consumed by the reaction, and becomes the 5′-end of the second strand product (also known as the “coding” or “sense” strand). FA is displaced during final “non-sense” strand synthesis and does not participate in subsequent rounds of amplification.

A standard thermocycle program is used to amplify the double stranded DNA product. Briefly, the reaction temperature is raised to 90-95° C. to denature the nucleic acids. The temperature is then lowered to 50-65° C. to allow primers to hybridize to complementary priming sites, then the temperature is raised to about 68-74° C. to allow the thermostabile DNA polymerase to extend hybridized primers along the template DNA molecules. The time for each temperature is between about 15 seconds and 2 minutes. These steps are then repeated between about 25 and 35 times to amplify double stranded DNA.

After the first denaturation cycle, the FA/FP complex hybridizes to the first strand cDNA and is extended by DNA polymerase to produce the second strand of the cDNA and yield a double stranded cDNA product. It is important to note that FUP and RUP do not match the initial RNA sequence, and thus will not participate in the reaction until after first and second strand cDNA synthesis has occurred.

First and second strand priming and synthesis, and final amplicon priming and synthesis, consume the FP and RP. Extension of the priming groups, typically 3′-OH, by the polymerase cause FP and RP to become covalently incorporated into the product cDNA ends. Incorporation of the FP and RP stem sequences as the 5′ ends of the cDNA provides the priming site for FUP and RUP, respectively, to bind and prime subsequent cDNA amplification. FUP and RUP are consumed by the reaction to produce the final double stranded DNA amplicon (FIG. 3). In addition, FP, RP, FUP, and/or RUP may have attached “detection” moieties to facilitate detection and/or capture of the cDNA or final DNA amplification product(s). Suitable “detection” moieties include enzymatic labels, fluorescent labels and radiolabels.

A preformed library of oligonucleotide primers is provided which comprises a first set of oligonucleotide primers and a second set of oligonucleotide primers, the primers having stem regions that allow coupling of the primers to form an amplification primer pair.

By conceptually separating the amplification primer into two pieces, a comprehensive amplification primer library can be prepared in advance, rather than synthesizing a plurality of amplification primers as needed. This primer pair system is advantageous to a single primer system because of increased binding affinity. A complete library of every possible 17-mer oligonucleotide, using the four natural bases, would consist of 417 (or about 1.7×10¹⁰) molecules. By providing the primers in two components, for example a library of 8-mers and a library of 9-mers, assembled quickly as needed, the size of the library needed is reduced to 4⁸+4⁹, or 327,650 molecules. The required complexity of the library is still further reduced by substituting one or more universal or degenerate bases for some of the natural bases. Thus, for example, if the 9-mer library consists of 5 universal bases followed by 4 natural bases, the number of components drops to 4⁴ (256), and the total library size is reduced to 4⁸+4⁴, about 66,000 molecules. The library complexity can also be reduced by dividing the antisense molecule into three or more segments. It is possible to synthesize and maintain libraries of this size, and rapidly assemble any desired primer pairs without the need for custom, de novo synthesis of long oligomers. Thus, one embodiment of the invention is a library comprising at least two sets of oligomers, wherein oligomers are selected from each set and coupled as needed.

At least one of the binding domains comprises about 3 to about 24 bases, preferably about 6 to about 8 bases. For example, a library can be constructed having a set of 6-mer binding domains, each of which binds only a single 6-mer sequence, and a set of 8-mer binding domains, in which only four of the bases are sequence-specific, and the remaining bases are degenerate or universal. The first set contains a possible 4₆ (4096) sequences, while the second set contains only 4⁴ (256) sequences (assuming 4 “specific” bases and 4 universal bases). By combining oligomers selected from the first and second sets as needed, 4¹⁰ (10⁶) different sequences can be generated using only 4,352 molecules. In contrast, a complete library of 14-mers would require 414 (2.7×10⁸) molecules.

The first oligonucleotide of the two-component primer (primer component P) comprises only naturally-occurring phosphodiester linkages and may contain one or more modified bases (for example, universal and degenerated bases as defined below). The second oligonucleotide (anchor component A) is modified to ensure high affinity binding to the RNA target sequence and contains one or more modified bases and modified backbone linkages, for example phosphorothioates, deoxy phosphorothioates, 2′-O-substituted phosphodiesters and deoxy analogs, 2′-O-substituted phosphodiesters and deoxy analogs, 2′-O-substituted phosphorothioates and deoxy analogs, morpholino, peptide nucleic acids (PNA; see U.S. Pat. No. 5,539,082), 2′-O-alkyl methylphosphonates, 3′-amidates, MMI, alkyl ethers (see Cook et al., U.S. Pat. No. 5,223,618) and others as described in Cook et al., U.S. Pat. No. 5,378,825, Sanghvi et al. (U.S. Pat. No. 5,489,677), Cook et al. (U.S. Pat. No. 5,541,307) and the like.

Universal bases suitable for use in the present invention include, but are not limited to, deoxy 5-nitroindole, deoxy 3-nitropyrrole, deoxy 4-nitrobenzimidazole, deoxy nebularine, deoxyinosine, 2′-OMe inosine, 2′-OMe 5-nitroindole, 2′-OMe 3-nitropyrrole, 2′-F inosine, 2′-F nebularine, 2′-F 5-nitroindole, 2′-F 4-nitrobenzimidazole, 2′-F 3-nitropyrrole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitrobenzimidazole, 2′-O-methoxyethyl 3-nitropyrrole, deoxy Rₚ MP-5-nitroindole dimer and 2′-OMe Rₚ MP-5-nitroindole dimer.

Degenerate bases suitable for use in the present invention include, but are not limited to, deoxy P (A & G), deoxy K (U & C), 2′-OMe 2-aminopurine (U & C), 2′-OMe P (G & A), 2′-OMe K (U & C), 2′-F-2-aminopurine (U & C), 2′-F P (G & A), 2′-F K (U & C), PNA-2-aminopurine (U & C), PNA-P (G & A), PNA-K (U & C), morpholino-2-aminopurine (U & C), morpholino-P (G & A), morpholino-K (U & C), phosphoramidate-2-aminopurine (C&U), phosphoramidate-P (G & A), phosphoramidate-K (U & C), 2′-O-methoxyethyl 2-aminopurine (U & C), 2′-O-methoxyethyl P (G & A), 2′-O-methoxyethyl K (U & C), deoxy Rₚ MP-KP dimer, deoxy Rₚ MP-PK dimer, deoxy Rₚ MP-KK dimer, deoxy Rₚ MP-PP dimer, 2′-OMe Rₚ MP-KP dimer, 2′-OMe Rₚ MP-PK dimer, 2′-OMe Rₚ MP-KK dimer and 2′-OMe Rₚ MP-PP dimer.

The coupling moieties are selected to join two oligomers from different sets by either covalent or non-covalent interaction, for example a non-covalent binding pair. The coupling moieties are preferably selected such that the coupling moiety present on oligonucleotides of the first set in a library do not couple with each other, but bind readily with coupling moieties on oligonucleotides of the second set, thus ensuring that the oligonucleotides couple in the intended orientation. In one embodiment, the coupling moieties are complementary oligonucleotides or oligonucleotide analogs.

### EXAMPLE 1

Amplification of PKCα

In the sequences shown below, L=linker from Glen Research (spacer 18, cat. #10-1918-90 or spacer 9, cat. #10-1909-90), underlined lower case nucleotides are gene-specific sequences and underlined upper case nucleotides are stem sequence regions. In this example, all primers contain only unmodified DNA backbones, and contain some high-affinity modified bases, such as C-5 Propynyl-C and C-5-propynyl-U. Anchors contain 2′-OMe-modified sugar backbones and C-5-Propynyl-C. Gene-specific regions for this example range in total length from 12 bases (6 each from anchors and primers) to 24 bases (12 each from anchors and primers). The step region is 24 bases long, and the universal primers (FUP and RUP) are 19 or 14 bases long.

| | | |
|---|---|---|
| FUP 100: | 5′-GCCACCTGTGGTCCACCTG-3′ | (SEQ ID NO: 1) |
| FUP 112: | 5′-GCCACCTGTGGTCC-3′ | (SEQ ID NO: 2) |
| FP 104: | 5′-GCCACCTGTGGTCCCACCTGCTGAG gtagaa-3′ | (SEQ ID NO: 3) |
| FP 106: | 5′-GCCACCTGTGGTCCACCTGCTGAG gtagaaatctgg-3′ | (SEQ ID NO: 4) |
| FA 108: | 5′-ctgtct (L) CTCAGCAGGT-3′ | (SEQ ID NO: 5) |
| FA 110: | 5′-cgacgactgtct (L) CTCAGCAGGT-3′ | (SEQ ID NO: 6) |
| RUP 101: | 5′-CACTTGTGGCCCAGATAGG-3′ | (SEQ ID NO: 7) |
| RUP 113: | 5′-CACTTGTGGCCCAG-3′ | (SEQ ID NO: 8) |
| RP 105: | 5′-CACTTGTGGCCCAGATAGGAGGCT gcactc-3′ | (SEQ ID NO: 9) |
| RP 107: | 5′-CACTTGTGGCCCAGATAGGAGGCT gcactccacgtc-3′ | (SEQ ID NO: 10) |
| RA 109: | 5′-catggt (L) AGCCTCCTAT-3′ | (SEQ ID NO: 11) |
| RA 111: | 5′-ttctaccatggt (L) AGCCTCCTA-3′ | (SEQ ID NO: 12) |

Total cellular or poly(A+) RNA (0.1-2,000 ng) is added in the presence of (per reaction):

10 mM Tris-HCl, pH 8.3

50 mM KCl

200 μM each dATP, dGTP, dCTP, dTTP

0.2-1.0 μM FUP

0.2-1.0 μM RUP

0.02-0.1 μM each gene-specific FA, FP, RA and RP

200 U/100 μl reverse transcriptase

200 U/100 μl thermostabile DNA polymerase

PCR is performed using the parameters described above. The size of the final product is verified by 1% agarose gel electrophoresis. The amplified PCR fragment is cleaned and purified using a commercial PCR cleaning kit (e.g., Qiagen), and can be used for in vitro or in vivo transfection of cells or tissues.

Although the examples described herein refer to amplification primer pairs for PCR amplification of a nucleic acid sequence, these primer pairs can be used for any DNA or RNA polymerase priming activity. For example, these primers pairs can be used in non-thermocycling primer-dependent amplification technologies that are used to compete commercially with PCR, such as strand displacement amplification (SDA) (Walker, PCR Methods Appl. 3:1-6, 1993; Spargo et al., Mol. Cell. Probes 10:247-256, 1996), self-sustained sequence replication (3 SR) and in situ self-sustained sequence replication (IS-3SR) (Mueller et al., Histochem. Cell Biol. 108:431-437, 1997), rolling circle amplification (RCA) (Lizardi et al., Nature Genet. 19:225-232, 1998), and nucleic acid sequence based amplification (NASBA) (Heim et al., Nucl. Acids Res. 26:2250-2251, 1998; Romano et al., Immunol. Invest. 26:15-28, 1997).

While particular embodiments of the invention have been described in detail, it will be apparent to those skilled in the art that these embodiments are exemplary rather than limiting, and the true scope of the invention is that defined in the following claims.

What is claimed is:

1. An amplification primer pair comprising an oligonucleotide anchor and primer, said anchor having a nucleic acid chemistry which is not a substrate for reverse transcriptases or DNA polymerases, and/or having a 3′-end which is not capable of priming nucleic acid synthesis;
wherein said primer has a nucleic acid chemistry that is a substrate for reverse transcriptases or DNA polymerases; and
wherein said anchor and said primer each include a region of complementary nucleotides which readily associate with each other to form a stem structure in the absence of a target nucleic acid, wherein the stem structure includes a region which is complementary to a universal primer.

2. The primer pair of claim 1, wherein said anchor sequence and said stem regions are connected by a flexible linker.

3. The primer pair of claim 2, wherein said flexible linker is selected from the group consisting of polyethylene glycol, polypropylene glycol, polyethylene, polypropylene, polyamides and polyesters.

4. The primer pair of claim 1, wherein said primer comprises a tail region which extends beyond the length of said stem region of said anchor.

5. The primer pair of claim 1, wherein said primer comprises one or more modified bases.

6. The primer pair of claim 1, wherein said anchor comprises one or more modified backbone linkages.

7. The primer pair of claim 1, wherein said anchor and said primer are each between 6 and 24 bases in length.

8. The primer pair of claim 1, further in association with a universal primer which is complementary to a region of said stem structure.

9. A sequencing primer pair comprising an oligonucleotide anchor and primer, said anchor having a nucleic acid chemistry which is not a substrate for reverse transcriptases or DNA polymerases, and/or having a 3′-end which is not capable of priming nucleic acid synthesis;
wherein said primer has a nucleic acid chemistry that is a substrate for reverse transcriptases or DNA polymerases; and
wherein said anchor and said primer each include a region of complementary nucleotides which readily associate with each other to form a stem structure in the absence of a target nucleic acid.

10. The primer pair of claim 9, wherein said stem structure includes a region which is complementary to a universal primer.

11. The primer pair of claim 10, wherein said anchor sequence and said stem region are connected by a flexible linker.

12. The primer pair of claim 11, wherein said flexible linker is selected from the group consisting of polyethylene glycol, polypropylene glycol, polyethylene, polypropylene, polyamides and polyesters.

13. The primer pair of claim 10, wherein said primer comprises a tail region which extends beyond the length of said stem region of said anchor.

14. The primer pair of claim 9, wherein said primer comprises one or more modified bases.

15. The primer pair of claim 9, wherein said anchor comprises one or more modified backbone linkages.

16. The primer pair of claim 9, wherein said anchor and said primer are each between 6 and 24 bases in length.

17. The primer pair of claim 9, further in association with a universal primer which is complementary to a region of said stem structure.

18. A method for amplifying a target nucleic acid sequence, comprising the steps of: combining said target nucleic acid sequence a forward anchor (FA), forward primer (FP), reverse anchor (RA), reverse primer (RP), forward universal primer (FUP) and reverse universal primer (RUP), wherein said FA/FP readily associate to form a first primer pair and said RA/RP readily associate to form a second primer pair via association of their complementary stem regions in the absence of said target nucleic acid, wherein said FUP is complementary to the FA/FP stem region, and wherein said RUP is complementary to the RAIRP stem region wherein said primer pairs are selected on the basis of complementarity to said target nucleic acid sequence to flank said target nucleic acid sequence; and
amplifying said nucleic acid sequence via enzyme-mediated amplification.

19. The method of claim 18, wherein said nucleic acid sequence encodes a therapeutic gene product.

20. The method of claim 18, wherein said nucleic acid sequence is DNA or RNA.

21. The method of claim 18, wherein said enzyme-mediated amplification is PCR amplification.

22. A method for amplifying a target nucleic acid sequence, comprising:
providing a first oligonucleotide primer comprising a first target binding region configured to bind to a first end of said target nucleic acid sequence, and a first stem region, wherein said first stem region comprises a first universal primer binding region;
providing a second oligonucleotide primer comprising a second target binding region configured to bind to said first end of said target nucleic acid sequence, and a second stem region that is homologous with said first stem region, so that said first oligonucleotide primer and said second oligonucleotide primer join together to form a first primer pair that binds to said target nucleic acid sequence;
providing a universal primer configured to bind with said first universal primer binding region;
providing an additional primer configured to bind with a second end of said target nucleic acid sequence; and
incubating said first primer pair, universal primer, and additional primer in the presence of said target nucleic acid sequence under conditions that amplify said target nucleic acid.

23. The method of claim 22, wherein the first stem region and the first target binding region are connected by a flexible linker.

24. The method of claim 23, wherein the flexible linker is selected from the group consisting of polyethylene glycol, polypropylene glycol, polyethylene, polypropylene, polyamides and polyesters.

25. The method of claim 22, wherein the second stem region and the second target binding region are connected by a flexible linker.

26. The method of claim 25, wherein the flexible linker is selected from the group consisting of polyethylene glycol, polypropylene glycol, polyethylene, polypropylene, polyamides and polyesters.

27. The method of claim 22, wherein the first oligonucleotide primer comprises one or more modified bases.

28. The method of claim 22, wherein the second oligonucleotide primer comprises one or more modified bases.

29. The method of claim 22, wherein the target nucleic acid sequence is DNA.

30. The method of claim 22, wherein the target nucleic acid sequence is RNA.

31. The method of claim 22, wherein said incubating comprises the Polymerase Chain Reaction.

32. The method of claim 22, wherein said amplifying is mediated by an enzyme selected from the group consisting of: Taq polymerase and reverse transcriptase.

33. The method of claim 22, wherein said additional primer comprises:
a third oligonucleotide primer comprising a third target binding region configured to bind to a second end of said target nucleic acid sequence, a reverse transcriptase binding region, and a third stem region, wherein said third stem region comprises a second universal primer binding region; and
a fourth oligonucleotide primer comprising a fourth target binding region configured to bind to said second end of said target nucleic acid sequence, and a fourth stem region that is homologous with said third stem region, so that said third oligonucleotide primer and said fourth oligonucleotide primer join together to form a second primer pair that binds to said target nucleic acid sequence.

34. A method of amplifying a target nucleic acid sequence, comprising the steps of:
providing a target nucleic acid sequence having a first amplification region and a second amplification region;
selecting from a library of first oligonucleotide primers, a first oligonucleotide primer that comprises a first target binding region that is homologous with a portion of the first amplification region, wherein said first oligonucleotide primer comprises a first stem region and a first universal primer binding region;
selecting from a library of second oligonucleotide primers, a second oligonucleotide primer that comprises a second target binding region that is homologous with a second portion of the first amplification region, wherein said second oligonucleotide primer comprises a second stem region that is homologous with said first stem region, so that said first oligonucleotide primer and said second oligonucleotide primer join together to form a first primer pair that binds to said target nucleic acid sequence at said first amplification region;
providing a universal primer configured to bind with said first universal primer binding region;
providing an additional primer configured to bind with a second amplification region of said target nucleic acid sequence; and
incubating said first primer pair, universal primer, and additional primer in the presence of said target nucleic acid sequence under conditions that amplify said target nucleic acid.

35. The method of claim 34, wherein the library of first oligonucleotide primers comprises greater than 65,536 oligonucleotides.

36. The method of claim 34, wherein the first target binding region comprises at least six nucleotides.

37. The method of claim 34, wherein the second target binding region comprises at least six nucleotides.

38. The method of claim 34, wherein the first target binding region and the first stem region are connected by a flexible linker.

39. The method of claim 38, wherein the flexible linker is selected from the group consisting of polyethylene glycol, polypropylene glycol, polyethylene, polypropylene, polyamides and polyesters.

40. The method of claim 34, wherein the second target binding region and the second stem region are connected by a flexible linker.

41. The method of claim 40, wherein the flexible linker is selected from the group consisting of polyethylene glycol, polypropylene glycol, polyethylene, polypropylene, polyamides and polyesters.

42. The method of claim 34, wherein the first oligonucleotide primer comprises one or more modified bases.

43. The method of claim 34, wherein the second oligonucleotide primer comprises one or more modified bases.

44. The method of claim 34, wherein the first oligonucleotide primer comprises one or more modified backbone linkages.

45. The method of claim 34, wherein the second oligonucleotide primer comprises one or more modified backbone linkages.

46. The method of claim 34, wherein the target nucleic acid sequence is DNA.

47. The method of claim 34, wherein the target nucleic acid sequence is RNA.

48. The method of claim 34, wherein said incubating comprises the Polymerase Chain Reaction.

49. The method of claim 34, wherein said amplifying is mediated by an enzyme selected from the group consisting of Taq polymerase and reverse transcriptase.


SEQUENCE LISTING (12 sequences)

SEQ ID NO: 1 - DNA, 19 bp, Artificial Sequence
  Synthetic oligonucleotide primers
gccacctgtggtccacctg

SEQ ID NO: 2 - DNA, 14 bp, Artificial Sequence
  Synthetic oligonucleotide primers
gccacctgtggtcc

SEQ ID NO: 3 - DNA, 31 bp, Artificial Sequence
  Synthetic oligonucleotide primers
gccacctgtggtcccacctgctgaggtagaa

SEQ ID NO: 4 - DNA, 36 bp, Artificial Sequence
  Synthetic oligonucleotide primers
gccacctgtggtccacctgctgaggtagaaatctgg

SEQ ID NO: 5 - DNA, 16 bp, Artificial Sequence
  Synthetic oligonucleotide primers
ctgtctctcagcaggt

SEQ ID NO: 6 - DNA, 22 bp, Artificial Sequence
  Synthetic oligonucleotide primers
cgacgactgtctctcagcaggt

SEQ ID NO: 7 - DNA, 19 bp, Artificial Sequence
  Synthetic oligonucleotide primers
cacttgtggcccagatagg

SEQ ID NO: 8 - DNA, 14 bp, Artificial Sequence
  Synthetic oligonucleotide primers
cacttgtggcccag

SEQ ID NO: 9 - DNA, 30 bp, Artificial Sequence
  Synthetic oligonucleotide primers
cacttgtggcccagataggaggctgcactc

SEQ ID NO: 10 - DNA, 36 bp, Artificial Sequence
  Synthetic oligonucleotide primers
cacttgtggcccagataggaggctgcactccacgtc

SEQ ID NO: 11 - DNA, 16 bp, Artificial Sequence
  Synthetic oligonucleotide primers
catggtagcctcctat

SEQ ID NO: 12 - DNA, 21 bp, Artificial Sequence
  Synthetic oligonucleotide primers
ttctaccatggtagcctccta